(19) United States
(12) Patent Application Publication
(10) Pub. No.: US 2021/0095258 A1
(43) Pub. Date: Apr. 1, 2021

(54) POST PARTUM TISSUE-DERIVED INDUCED PLURIPOTENT STEM CELLS AND USES THEREOF

(71) Applicant: Celularity Inc., Warren, NJ (US)

(72) Inventors: James EDINGER, Belford, NJ (US); Robert J. HARIRI, Bernardsville, NJ (US); Sourav SINHA, New York, NY (US); Xiaokui ZHANG, Martinsville, NJ (US)

(73) Assignee: Celularity Inc., Warren, NJ (US)

(21) Appl. No.: 16/971,936

(22) PCT Filed: Feb. 22, 2019
(86) PCT No.: PCT/US2019/019311

§ 371 Date: Aug. 21, 2020

Related U.S. Application Data
(60) Provisional application No. 62/634,103, filed on Feb. 22, 2018.

(51) Int. Cl. C12N 5/074

(52) U.S. Cl. CPC C12N 5/0696; C12N 2506/025; C12N 2501/603; C12N 2501/608; C12N 2501/604; C12N 2501/606; C12N 2501/605; C12N 2501/602

(57) ABSTRACT

The present invention provides an induced pluripotent stem (iPS) cell, or population of iPS cells, wherein the cell or cells giving rise to the iPS cell(s) are obtained from human postpartum tissue or cells, wherein the iPS cell(s) have increased levels of one or more factors selected from Group I: an Oct family member, a Sox family member, a Klf family member, a Myc family member, Nanog, Lin28, and combinations thereof. The present invention also provides differentiated cells derived from the cells of the invention and compositions, including pharmaceutical compositions comprising the cells of the invention. The invention further provides uses of the cells of the invention, e.g., in the treatment of a subject suffering from a disease of disorder. The invention additionally provides methods of generating iPS cell(s) from postpartum tissue, such as the cells of the invention.

This application claims priority benefit of U.S. Patent Application No. 62/634,103, filed Feb. 22, 2018, the contents of which are incorporated herein by reference in their entirety.

### 1. FIELD

The present invention relates, in part, to the generation of induced pluripotent stem (iPS) cells from postpartum tissue. For example, the present invention relates to the generation of iPS cells from maternal and non-maternal cells and from specific populations of postpartum tissue-derived cells.

### 2. BACKGROUND

The generation of IPS cells, cells which have been modified to restore pluripotency to previously differentiated cells, shows a great deal of promise for providing the benefits of stem cell therapy without the ethical concerns surrounding embryonic stem cells. IPS cells have been generated, for example from adult human somatic cells by expression of the four factors OCT4, SOX2, KLF4, and c-MYC. Takahashi et al. (Cell 131: 861-872 (2007)). Similarly, iPS cells also have been generated from somatic cells by expression of the factors OCT4 and SOX2 plus NANOG and LIN28. Yu et al. (Science 318: 1917-1920 (2007)). However, there exists an unmet need for alternative methods of generating iPS cells which can produce cells from alternative and sources, ideally with less genetic modification from the iPS generation process or from the deleterious effects of aging that can accompany the use of adult cells. The present invention is directed, in part to addressing these unmet needs.

### 3. SUMMARY

The present invention is directed towards iPS cells derived from postpartum tissue, e.g., iPS cells derived from umbilical cord, umbilical cord blood or placenta. The inventors have recognized that the postpartum tissue provides a source of both non-maternal cells which have not been subject to the deleterious genetic effects of aging, and a valuable source of less-committed progenitor cells that can be difficult to obtain and/or invasive to obtain from a subject by alternative means. Use of the tissues and cells of the invention is expected to provide iPS cells with quantifiable improved characteristics and, ultimately, greater utility than current iPS cells. Similarly, the inventors have conceived that alternative methods of inducing pluripotency, e.g., genes induced and methods of induction, will likewise improve the quality and utility of available iPS cells.

In one aspect, provided herein is an induced pluripotent stem (iPS) cell, or population of iPS cells, wherein the cell or cells giving rise to the iPS cell(s) are obtained from human postpartum tissue or cells, wherein the iPS cell(s) have increased levels of one or more factors selected from Group I: an Oct family member, a Sox family member, a Klf family member, a Myc family member, Nanog, Lin28, and combinations thereof.

In another aspect, provided herein is a population of human cells which is differentiated from the iPS cell or population of iPS cells of the invention.

In another aspect, provided herein is the use of the iPS cell or population of iPS cells of the invention or the population of human cells which is differentiated from the iPS cell(s) of the invention for use in treatment of a disease or disorder in a human subject or in the manufacture of a medicament for the treatment of a disease or disorder in a human subject.

In another aspect, provided herein is a method of treatment of a disease or disorder in a human subject comprising the step of: administering to the subject a therapeutically effective amount of the population of iPS cells of the invention or the population of human cells which is differentiated from the iPS cell(s) of the invention, wherein the therapeutically effective amount is an amount of cells sufficient to reduce or eliminate the disease or disorder, or a symptom thereof, in the subject.

In another aspect, provided herein is a method of vaccinating a subject against a cancer comprising the step of: administering to the subject a therapeutically effective amount of a vaccine comprising the population of irradiated iPS cells of the invention, wherein the therapeutically effective amount is an amount of vaccine sufficient elicit an immune response to a cancer-associated antigen present in the vaccine in the subject.

Administration and delivery of cells, e.g., for the purpose of providing cells of the present invention to a subject, can include any method of parenteral administration, including intravenous infusion, direct intramuscular, subcutaneous, intracompartmental, intraperitoneal, and subdermal administration. The dose and formulation of said cells can also include any conventional means of suspending and injecting said product, including those provided elsewhere herein. In a specific embodiment, the cells are administered to a subject in need thereof.

In another aspect, provided herein is a composition comprising the population of iPS cells of the invention or the population of human cells of the invention, or a combination thereof; and a pharmaceutical composition comprising the population of iPS cells of the invention or the population of human cells of the invention, or a combination thereof and a pharmaceutically acceptable carrier.

In another aspect, provided herein is a method of generating an iPS cell or population of iPS cells, the method comprising the steps of: i) obtaining a sample of cells from human postpartum tissue; ii) inducing increased levels of one or more factors selected from Group I: an Oct family member, a Sox family member, a Klf family member, a Myc family member, Nanog, Lin28, and combinations thereof in at least one cell of step i); and iii) selecting an embryonic stem (ES) cell-like cell or ES cells from the cells of step ii); thereby generating the iPS cell or population of iPS cells.

### 4. DETAILED DESCRIPTION

### 4.1 Definitions

All patents, applications, published applications and other publications are incorporated by reference in their entirety. Unless defined otherwise, all technical and scientific terms used herein have the same meaning as is commonly understood by one of ordinary skill in the art. All patents, applications, published applications and other publications are incorporated herein by reference in their entirety. In the event that there is a plurality of definitions for a term herein, those in this section prevail unless stated otherwise.

The term “about” or “approximately” means within 20%, within 10%, within 5%, within 1% or less of a given value or range.

As used herein, “administer” or “administration” refers to the act of injecting or otherwise physically delivering a substance as it exists outside the body (e.g., a PDSC or other stem cell provided herein) into a patient. Delivery, for example, can occur by any method including, but not limited to, intradermal, intravenous, intramuscular, subcutaneous delivery and/or any other method of physical delivery described herein or known in the art.

The term “autologous” as used herein refers to organs, tissues, cells, fluids or other bioactive molecules that are reimplanted in the same individual that they originated from.

As used herein, the term “composition” is intended to encompass a product containing the specified ingredients (e.g., PDSC or other stem cell provided herein) and, optionally, in the specified amounts, as well as any product which results, directly or indirectly, from combination of the specified ingredients in, optionally, the specified amounts.

The term “culturing” as used herein refers to propagating or cultivation of a cell, a population of cells, a tissue, or an organ, by incubating in an environment under conditions and for a period of time sufficient to support cell propagation or viability. Culturing can include expanding or proliferating a cell or population of cells, such as iPS cells.

The term “perfuse” or “perfusion” as used herein refers to the act of pouring or passaging a fluid over or through an organ or tissue, preferably the passage of fluid through an organ or tissue with sufficient force or pressure to remove any residual cells, e.g., non-attached cells from the organ or tissue. As used herein, the term “perfusate” refers to the fluid collected following its passage through an organ or tissue. In a preferred embodiment, the perfusate contains one or more anticoagulants.

The term “progenitor cell” as used herein refers to a cell that is committed to differentiate into a specific type of cell or to form a specific type of tissue.

The term “effective amount” as used herein refers to the amount of a therapy (e.g., stem cells, such as iPS cells, or a population of stem cells, such as iPS cells, as provided herein) which is sufficient to achieve a desired result or specified outcome. In some embodiments, the effective amount is an amount sufficient to reduce and/or ameliorate the severity and/or duration of a given disease, disorder or condition (e.g., aging) and/or a symptom related thereto. This term also encompasses an amount necessary for the reduction or amelioration of the advancement or progression of a given disease, disorder or condition (e.g., aging), reduction or amelioration of the recurrence, development or onset of a given disease, disorder or condition (e.g., aging). In some embodiments, the effective amount of a population of iPS cells provided herein is from about 1×10⁵ to about 1×10¹¹, e.g., about 3×10⁵, 5×10⁵, 1×10⁶, 3×10⁶, 5×10⁶, 1×10⁷, 3×10⁷, 5×10⁷, 1×10⁸, 2×10⁸, 3×10⁸, 4×10⁸, 5×10⁸, 6×10⁸, 7×10⁸, 8×10⁸, 8×10⁸, 1×10⁹, 2×10⁹, 3×10⁹, 4×10⁹, 5×10⁹, 1×10¹⁰, 5×10¹⁰, or 1×10¹¹ (or any range therein). In some embodiments, “effective amount” as used herein also refers to the amount of a population of iPS cells provided herein to achieve a specified result.

The term “postpartum tissue” as used herein refers to tissues including the amnion, the amniotic fluid, the placenta or any part thereof, or the umbilical cord or any part thereof. Nonlimiting examples of postpartum tissue as used herein include cord blood, Wharton's jelly, umbilical cord tissue, placental blood, placental perfusate, amniotic fluid, chorion, decidua or a placental lobe or cotyledon.

A placenta has the genotype of the fetus that develops within it, but is also in close physical contact with maternal tissues during gestation. As such, as used herein, the terms “non-maternal” or “fetal genotype” means derived from the fetus or having the genotype of the fetus, e.g., the genotype of the fetus associated with the placenta from which particular isolated placental cells, as described herein, are obtained, as opposed to the genotype of the mother that carried the fetus. As used herein, the term “maternal genotype” or “maternal” means the genotype of the mother that carried the fetus, e.g., the fetus associated with the placenta from which particular isolated placental cells, as described herein, are obtained.

The terms “generate,” “generation” and “generating” as used herein refer to the production of new cells in a subject and optionally the further differentiation into mature, functioning cells.

As used herein, “isolating” a cell (e.g., a iPS cell) refers to a process of dissociating or otherwise removing a cell from a tissue sample (e.g., placental tissue), and separating the cells from other cells or non-cells in the tissue. Isolated cells will generally free from contamination by other cell types and will generally be able to be propagated and expanded.

As used herein, the term “isolated cell,” e.g., “isolated stem cell,” means a cell that is substantially separated from other, different cells of the tissue, e.g., placenta, from which the stem cell is derived. A stem cell is “isolated” if at least 50%, 60%, 70%, 80%, 90%, 95%, or at least 99% of the cells with which the population of cells, or cells from which the population of cells is derived, is naturally associated, i.e., stem cells displaying a different marker profile, are removed from the stem cell, e.g., during collection and/or culture of the stem cell. In some embodiments, an isolated cell exists in the presence of a small fraction of other cell types that do not interfere with the utilization of the cell for analysis, production or expansion of the cells. A population of isolated cells can be at least 30%, 40%, 50%, 60%, 70%, 80%, 85%, 90%, 95%, 98%, or 99% pure, or any interval thereof. In a specific embodiment, a population of isolated cells are at least 98% or at least 99% pure. As used herein, the term “population of isolated cells” means a population of cells that is substantially separated from other cells of a tissue, e.g., placenta, from which the population of cells is derived.

The terms “optional” or “optionally” as used herein means that the subsequently described event or circumstance may or may not occur, and that the description includes, without limitation, instances where said event or circumstance occurs and instances in which it does not.

The term “pharmaceutically acceptable” as used herein means being approved by a regulatory agency of the Federal or a state government, or listed in the U.S. Pharmacopeia, European Pharmacopeia or other generally recognized Pharmacopeia for use in animals, and more particularly in humans.

As used herein, a cell is “positive” for a particular marker when that marker is detectable above background. For example, a placental cell is positive for, e.g., CD73 because CD73 is detectable on placental cells in an amount detectably greater than background (in comparison to, e.g., an isotype control). A cell is also positive for a marker when that marker can be used to distinguish the cell from at least one other cell type, or can be used to select or isolate the cell when present or expressed by the cell. In the context of, e.g., antibody-mediated detection, “positive,” as an indication a particular cell surface marker is present, means that the marker is detectable using an antibody, e.g., a fluorescently-labeled antibody, specific for that marker; “positive” also refers to a cell exhibiting the marker in an amount that produces a signal, e.g., in a cytometer, that is detectably above background. For example, a cell is “CD200⁺” where the cell is detectably labeled with an antibody specific to CD200, and the signal from the antibody is detectably higher than that of a control (e.g., background or an isotype control). Conversely, “negative” in the same context means that the cell surface marker is not detectable using an antibody specific for that marker compared a control (e.g., background or an isotype control). For example, a cell is “CD34⁻” where the cell is not reproducibly detectably labeled with an antibody specific to CD34 to a greater degree than a control (e.g., background or an isotype control). Markers not detected, or not detectable, using antibodies are determined to be positive or negative in a similar manner, using an appropriate control. For example, a cell or population of cells can be determined to be OCT-4⁺ if the amount of OCT-4 RNA detected in RNA from the cell or population of cells is detectably greater than background as determined, e.g., by a method of detecting RNA such as RT-PCR, slot blots, etc. Unless otherwise noted herein, cluster of differentiation (“CD”) markers are detected using antibodies. In certain embodiments, OCT-4 is determined to be present, and a cell is “OCT-4⁺” if OCT-4 is detectable using RT-PCR.

As used herein, the term “SH2” refers to an antibody that binds an epitope on the marker CD105. Thus, cells that are referred to as SH2⁺ are CD105⁺.

As used herein, the terms “SH3” and “SH4” refer to antibodies that bind epitopes present on the marker CD73. Thus, cells that are referred to as SH3⁺ and/or SH4⁺ are CD73⁺.

As used herein, the term “stem cells” refers to cells that have the capacity to self-renew and to generate differentiated progeny. The term “pluripotent stem cells” refers to stem cells that has complete differentiation versatility, i.e., the capacity to grow into any of the fetal or adult mammalian body's approximately 260 cell types. For example, pluripotent stem cells have the potential to differentiate into three germ layers: endoderm (e.g., blood vessels), mesoderm (e.g., muscle, bone and blood) and ectoderm (e.g., epidermal tissues and nervous system), and therefore, can give rise to any fetal or adult cell type. The term “induced pluripotent stem cells” as used herein refers to differentiated mammalian somatic cells (e.g., adult somatic cells, such as skin) that have been reprogrammed to exhibit at least one characteristic of pluripotency. The term “multipotent stem cells” as used herein refers to a stem cell that has the capacity to grow into any subset of the fetal or adult mammalian body's approximately 260 cell types. For example, certain multipotent stem cells can differentiate into at least one cell type of ectoderm, mesoderm and endoderm germ layers. The term “embryonic stem cells” as used herein refers to stem cells derived from the inner cell mass of an early stage embryo, e.g., human, that can proliferate in vitro in an undifferentiated state and are pluripotent. The term “bone marrow stem cells” as used herein refers to stem cells obtained from or derived from bone marrow. The term “amniotic stem cells” as used herein refers to stem cells collected from amniotic fluid or amniotic membrane. The term “embryonic germ cells” as used herein refers to cells derived from primordial germ cells, which exhibit an embryonic pluripotent cell phenotype.

As used herein, the terms “subject” and “patient” are used interchangeably. As used herein, a subject can be a mammal such as a non-primate (e.g., cows, pigs, horses, cats, dogs, rats, etc.) or a primate (e.g., monkey and human). In specific embodiments, the subject is a human. In one embodiment, the subject is a mammal (e g., a human) having or at risk of developing a disease, disorder or condition. In some embodiments, the subject is a subject in need thereof.

As used herein, “treat,” “treatment” and “treating” encompass the cure of, remediation of, improvement of, lessening of the severity of, or reduction in the time course of, a disease, disorder or condition, or any parameter or symptom thereof.

### 4.2 Additional Embodiments

Other embodiments of the various methods described herein are provided below:

In one aspect, provided herein is an induced pluripotent stem (iPS) cell, or population of iPS cells, wherein the cell or cells giving rise to the iPS cell(s) are obtained from human postpartum tissue or cells, wherein the iPS cell(s) have increased levels of one or more factors selected from Group I: an Oct family member, a Sox family member, a Klf family member, a Myc family member, Nanog, Lin28, and combinations thereof.

In some aspects of the invention, a single factor selected from Group I is increased. In other aspects of the invention, 2, 3, 4, or more factors selected from Group I are increased.

In some aspects of the invention, the Oct family member is selected from the group consisting of Oct3, Oct3/4, and Oct4. In preferred aspects, the Oct family member is Oct4. In some aspects of the invention, the Sox family member is selected from the group consisting of Sox1, Sox2, Sox3, Sox15, and Sox18. In preferred aspects, Sox family member is Sox2. In some aspects of the invention, the Klf family member is selected from the group consisting of Klf1, Klf2, Klf4, and Klf5. In preferred aspects, the Klf family member is Klf4. In some aspects of the invention, the wherein the Myc family member is selected from the group consisting of c-Myc, L-Myc, and N-Myc. In preferred aspects, the Myc family member is c-Myc.

In some aspects of the invention, the iPS cell(s) additionally have increased levels of one or more factors selected from Group II: C/EBPα, Nr5a2, Gls1, ESRRB, Sal4, Rex1, Myb, Kit, Gdf3, Ecat1, Dppa2, Dppa3, Dppa4, Dppa5, Fbox15, Eras, Dnmt31, Ecat8, Gdf3, Fthl17, Sall4, Utf1, Tcl1, and combinations thereof.

In some aspects of the invention, the iPS cell(s) comprise an exogenous nucleic acid encoding one or more of the factors from Group I or Group II. In some aspects of the invention the nucleic acid is a retroviral vector, a lentiviral vector, an episomal vector, or a transgene vector. In some of these aspects, the nucleic acid is introduced by a transfection method, e.g., a stable transfection method or a transient transfection method. In other aspects the nucleic acid is introduced by a transduction method.

In some aspects of the invention, the cell(s) are induced to express the one or more factors from Group I or Group II by the methods set forth herein, e.g., by treatment with a small molecule, by epigenetic modification, by contacting the cell with an RNA or by contacting the cell with a growth factor or cytokine. In other aspects the cell(s) are induced to express the one or more factors from Group I or Group II by use of a gene editing technology. In preferred aspects, the gene editing technology is selected from the group consisting of CRISPR, C2c2-RNA/CRISPR, Zinc Finger Nuclease (ZNF), TALEN, other gene editing techniques, or combinations thereof.

In some aspects of the invention, the iPS cell(s) are derived from cells obtained from the umbilical cord, preferably wherein the postpartum cells are selected from the group consisting of cord blood, Wharton's jelly, and umbilical cord tissue. In other aspects, the iPS cell(s) are derived from cells obtained from placental blood, placental perfusate, the amniotic membrane, amniotic fluid, the chorion, the decidua. In some aspects of the invention, the iPS cells are mononuclear cells, e.g., cells obtained by red blood cell lysis. In some aspects of the invention the iPS cell(s) are derived from maternal tissue, in other aspects they are derived from non-maternal tissue. In some aspects of the invention, the iPS cell(s) are derived from fresh cells, in other aspects they are derived from cryopreserved cells. In some of these aspects, the cells are obtained from a cord blood bank. In yet other aspects of the invention the iPS cells are derived from cells from a combination of the above tissues.

In some aspects of the invention, the iPS cell(s) are derived from cell populations which comprise cells having certain marker phenotypes. In some aspects, the populations comprise cells that are CD34⁺. In other aspects the populations comprise cells that are CD34⁻, CD10⁺, SH2⁺, and CD90⁺ placental multipotent cells; CD34⁻, CD38⁻, CD45⁻, CD10³⁰ , CD29⁺, CD44⁺, CD54⁺, CD90⁺, SH2⁺, SH3⁺, SH4⁺ and OCT-4⁺; CD34⁻, CD10⁺, CD105⁺, and CD200⁺; CD73⁺; CD73⁺ and CD105⁺; CD200⁺; CD34⁻, CD38⁻, CD45⁻, OCT-4⁺, and CD200⁺; CD34⁻, CD38⁻, CD34⁻, CD73⁺, OCT-4⁺, and CD200⁺; OCT-4⁺; CD73⁺, CD105⁺, and OCT-4⁺; CD73⁺, CD105⁺, and CD200⁺; CD200⁺ and OCT-4⁺; CD73⁺, CD105⁺, and HLA-G⁺; CD73⁺, CD105⁺, CD200⁺, and HLA-G⁺; CD34⁻, CD38⁻, CD45⁻, and HLA-G⁺; CD34⁻; CD38⁻; CD45⁻; CD34⁻ and CD38⁻; CD34⁻ and CD45⁻; CD38⁻ and CD45⁻; or CD34⁻, CD38⁻ and CD45⁻. In some of the above aspects, the populations consist essentially of cells of the indicated phenotype; in other aspects, the iPS cells are derived from cells having the indicated phenotypes.

In some aspects of the invention, the iPS cell(s) are derived from a sorted population of cells. In some of these aspects, the cells are sorted by magnetic-activated cell sorting (MACS); in some aspects they are sorted by is fluorescence-activated cell sorting (FACS).

In some aspects of the invention, the iPS cell(s) are characterized by identification of colonies of cells with ES cell- or embryoid body-like morphology. In some aspects of the invention, the iPS cell(s) are characterized by expression of one or more embryonic stem cell surface antigens. In preferred aspects, one of more of these antigens are selected from the group consisting of CD9, SSEA-1, SSEA-3, SSEA-4, tumor-related antigen (TRA)-1-60, TRA-1-81 and TRA-2-49/6E (alkaline phosphatase), and combinations thereof.

In some aspects of the invention, the iPS cell(s) are characterized by expression of one or more embryonic stem cell markers. In preferred aspects, one of more of these markers are selected from the group consisting of Group III: 5T4, ABCG2, Activin, RIB/ALK-4, Activin RIIB, Alkaline Phosphatase/ALPL, E-Cadherin, Cbx2, CD9, CD30/TNFRSF8, CD117/c-kit, CDX2, CHD1, Cripto, DNMT3B, DPPA2, DPPA4, DPPA5/ESG1, EpCAM/TROP1, ERR beta/NR3B2, ERVMER34-1, ESGP, F-box protein 15/FBXO15, FGF-4, FGF-5, FoxD3, GBX2, GCNF/NR6A1, GDF-3, Integrin alpha 6/CD49f, Integrin alpha 6 beta 1, Integrin alpha 6 beta 4, Integrin beta 1/CD29, KLF4, KLF5, L1TD1, Lefty, Lefty-1, Lefty-A, LIN-28A, LIN-28B, LIN-41, c-Maf, c-Myc, Nanog, Oct-3/4, Oct-4A, Oct-4B, Podocalyxin, Rex-1/ZFP42, Smad2, Smad2/3, SOX2, SSEA-1, SSEA-3, SSEA-4, STAT3, Stella/Dppa3, SUZ12, TBX2, TBX3, TBX5, TEX19, TEX19.1, THAP11, TRA-1-60(R), TRA-1-81, TROP-2, UTF1, VISTA/B7-H5/PD-1H, ZIC3, and combinations thereof. In some aspects of the invention, the iPS cell(s) are characterized by their methylation pattern. In preferred aspects, they are characterized by their methylation pattern at CpG residues.

In some aspects of the invention, the iPS cell(s) are characterized for pluripotency. In some of these aspects, pluripotency is verified by the ability to differentiate into each of mesoderm ectoderm and endoderm, e.g., in vitro or in vivo.

In another aspect, provided herein is a population of human cells which is differentiated from the iPS cell or population of iPS cells of the invention. In some of these aspects, cell is a population of cells from an organ or tissue selected from Group IV: digestive system, circulatory system, endocrine system, excretory system, immune system, integumentary system, muscular system, nervous system, reproductive system, respiratory system, skeletal system, lungs, liver, heart, brain, kidney, skin, bone, stomach, pancreas, bladder, gall bladder, small intestine, large intestine, prostate, testes, ovaries, spinal cord, pharynx, larynx, trachea, bronchi, diaphragm, ureter, urethra, esophagus, colon, thymus, spleen, and combinations thereof.

In another aspect, provided herein is the use of the iPS cell or population of iPS cells of the invention or the population of human cells which is differentiated from the iPS cell(s) of the invention for use in treatment of a disease or disorder in a human subject or in the manufacture of a medicament for the treatment of a disease or disorder in a human subject.

In some aspects of the invention, cell is a population of cells from an organ or tissue selected from Group IV: digestive system, circulatory system, endocrine system, excretory system, immune system, integumentary system, muscular system, nervous system, reproductive system, respiratory system, skeletal system, lungs, liver, heart, brain, kidney, skin, bone, stomach, pancreas, bladder, gall bladder, small intestine, large intestine, prostate, testes, ovaries, spinal cord, pharynx, larynx, trachea, bronchi, diaphragm, ureter, urethra, esophagus, colon, thymus, spleen, and combinations thereof.

In some aspects of the invention, the disease or disorder to be treated is selected from the group consisting of inflammatory diseases or disorders, infectious diseases or disorders, developmental diseases or disorders, metabolic diseases or disorders, autoimmune diseases or disorders, genetic diseases or disorders, mental diseases or disorders, behavioral diseases or disorders, cancers, mitochondrial diseases or disorders, cryptogenic diseases or disorders, environmental diseases or disorders, digestive system diseases or disorders, circulatory system diseases or disorders, endocrine system diseases or disorders, excretory system diseases or disorders, immune system diseases or disorders, integumentary system diseases or disorders, muscular system diseases or disorders, nervous system diseases or disorders, reproductive system diseases or disorders, respiratory system diseases or disorders, skeletal system diseases or disorders, lungs diseases or disorders, liver diseases or disorders, heart diseases or disorders, brain diseases or disorders, kidney diseases or disorders, skin diseases or disorders, bone diseases or disorders, stomach diseases or disorders, pancreas diseases or disorders, bladder diseases or disorders, gall bladder diseases or disorders, small intestine diseases or disorders, large intestine diseases or disorders, prostate diseases or disorders, testes diseases or disorders, ovaries diseases or disorders, spinal cord diseases or disorders, pharynx diseases or disorders, larynx diseases or disorders, trachea diseases or disorders, bronchi diseases or disorders, diaphragm diseases or disorders, ureter diseases or disorders, urethra diseases or disorders, esophagus diseases or disorders, colon diseases or disorders, thymus diseases or disorders, spleen diseases or disorders, and combinations thereof.

In another aspect, provided herein is a method of treatment of a disease or disorder in a human subject comprising the step of: administering to the subject a therapeutically effective amount of the population of iPS cells of the invention or the population of human cells which is differentiated from the iPS cell(s) of the invention, wherein the therapeutically effective amount is an amount of cells sufficient to reduce or eliminate the disease or disorder, or a symptom thereof, in the subject.

In another aspect, provided herein is a method of vaccinating a subject against a cancer comprising the step of: administering to the subject a therapeutically effective amount of a vaccine comprising the population of irradiated iPS cells of the invention, wherein the therapeutically effective amount is an amount of vaccine sufficient elicit an immune response to a cancer-associated antigen present in the vaccine in the subject.

In some aspects of the invention, the cancer is selected from the group consisting of a breast cancer, a lung cancer, a skin cancer, and a hematologic cancer. In preferred aspects, the cancer is a hematologic cancer, e.g., a cancer is selected from the group consisting of a leukemia, a lymphoma, and a myeloma.

In another aspect, provided herein is a composition comprising the population of iPS cells of the invention or the population of human cells of the invention, or a combination thereof; and a pharmaceutical composition comprising the population of iPS cells of the invention or the population of human cells of the invention, or a combination thereof and a pharmaceutically acceptable carrier.

In another aspect, provided herein is a method of generating an iPS cell or population of iPS cells, the method comprising the steps of: i) obtaining a sample of cells from human postpartum tissue; ii) inducing increased levels of one or more factors selected from Group I: an Oct family member, a Sox family member, a Klf family member, a Myc family member, Nanog, Lin28, and combinations thereof in at least one cell of step i); and iii) selecting an embryonic stem (ES) cell-like cell or ES cells from the cells of step ii); thereby generating the iPS cell or population of iPS cells.

In some aspects of the invention, inducing increased levels of one or more of the factors of Group I or Group II comprises contacting cell(s) of step i) with a nucleic acid encoding one or more of the factors from Group I or Group II. In some of these aspects, the nucleic acid is a viral vector (e.g., a retroviral vector or a lentiviral vector), a transgene vector, or an episomal vector. In other aspects of the invention the nucleic acid is a DNA or modified DNA, or an RNA or modified RNA. In some of these aspects the RNA or modified RNA is an mRNA, an miRNA, a piRNA, an siRNA, an shRNA, a srRNA, an snoRNA, a tsRNA, a U-RNA, and combinations thereof. In some of these aspects, the DNA is contacted with the cell by a transfection method, e.g., a stable or transient transfection method; in other aspects, the DNA is contacted with the cell by a transduction method.

In some aspects of the invention, the cell(s) are induced to express the one or more factors from Group I or Group II by use of a gene editing technology. In preferred aspects, the gene editing technology is selected from the group consisting of CRISPR, C2c2-RNA/CRISPR, Zinc Finger Nuclease (ZNF), TALEN, other gene editing techniques, or combinations thereof. In some aspects of the invention inducing increased levels of one or more of the factors of Group I or Group II comprises epigenetic modification; in other aspects the cell is contacted with a small molecule or with a cytokine or growth factor. In yet other aspects of the invention one or more factors from Group I or Group II are increased in expression by different means than that of one or more other factors from Group I or Group II, e.g., by combinations of the above means.

In another aspect, provided herein is a method of generating a population of human cells comprising differentiated cells, the method comprising the steps of: i) obtaining a population of iPS cells by the method of any one of claims 88-164; and ii) contacting the cells of step i) with conditions that support differentiation into a desired cell type, thereby generating the population of human cells comprising differentiated cells.

In some aspects of the invention, step ii) comprises culturing the cells under conditions that support differentiation into the desired cell type in vitro, in other aspects, step ii) comprises providing the cells to a desired site of differentiation in vivo.

In some aspects of the invention, the desired cell type is cells from an organ or tissue selected from Group IV: digestive system, circulatory system, endocrine system, excretory system, immune system, integumentary system, muscular system, nervous system, reproductive system, respiratory system, skeletal system, lungs, liver, heart, brain, kidney, skin, bone, stomach, pancreas, bladder, gall bladder, small intestine, large intestine, prostate, testes, ovaries, spinal cord, pharynx, larynx, trachea, bronchi, diaphragm, ureter, urethra, esophagus, colon, thymus, spleen, and combinations thereof.

### 5. EXAMPLES

### Example 1: Collection and Handling of Placenta

Generally, a human placenta is recovered shortly after its expulsion after birth. In one embodiment, the placenta is recovered from a patient after informed consent and after a complete medical history of the patient is taken and is associated with the placenta. In some embodiments, the medical history continues after delivery. Such a medical history can be used to coordinate subsequent use of the placenta or the stem cells harvested therefrom. For example, human postpartum cells can be used, in light of the medical history, for personalized medicine for the infant associated with the placenta, or for parents, siblings or other relatives of the infant.

Prior to recovery of placenta derived postpartum cells, the umbilical cord blood and placental blood are removed. In certain embodiments, after delivery, the cord blood in the placenta is recovered. The placenta can be subjected to a conventional cord blood recovery process. Typically a needle or cannula is used, with the aid of gravity, to exsanguinate the placenta (see, e.g., Anderson, U.S. Pat. No. 5,372,581; Hessel et al., U.S. Pat. No. 5,415,665). The needle or cannula is usually placed in the umbilical vein and the placenta can be gently massaged to aid in draining cord blood from the placenta. Such cord blood recovery may be performed commercially, e.g., LifeBank USA, Cedar Knolls, N.J., ViaCord, Cord Blood Registry and Cryocell. In some embodiments, the placenta is gravity drained without further manipulation so as to minimize tissue disruption during cord blood recovery.

Typically, a placenta is transported from the delivery or birthing room to another location, e.g., a laboratory, for recovery of cord blood and collection of stem cells by, e.g., perfusion or tissue dissociation. The placenta can be transported in a sterile, thermally insulated transport device (maintaining the temperature of the placenta between 20-28° C.), for example, by placing the placenta, with clamped proximal umbilical cord, in a sterile zip-lock plastic bag, which is then placed in an insulated container. In another embodiment, the placenta is transported in a cord blood collection kit substantially as described in pending U.S. Pat. No. 7,147,626. The placenta can be delivered to the laboratory four to twenty-four hours following delivery. In certain embodiments, the proximal umbilical cord is clamped, such as within 4-5 cm (centimeter) of the insertion into the placental disc prior to cord blood recovery. In other embodiments, the proximal umbilical cord is clamped after cord blood recovery but prior to further processing of the placenta.

The placenta, prior to stem cell collection, can be stored under sterile conditions and at either room temperature or at a temperature of 5 to 25° C. (centigrade). The placenta may be stored for a period of for a period of four to twenty-four hours, up to forty-eight hours, or longer than forty eight hours, prior to perfusing the placenta to remove any residual cord blood. In one embodiment, the placenta is harvested from between about zero hours to about two hours post-expulsion. The placenta can be stored in an anticoagulant solution at a temperature of 5 to 25° C. (centigrade). Suitable anticoagulant solutions are well known in the art. For example, a solution of heparin or warfarin sodium can be used. In one embodiment, the anticoagulant solution comprises a solution of heparin (e.g., 1% w/w in 1:1000 solution). The exsanguinated placenta is typically stored for no more than 36 hours before postpartum cells are collected.

The mammalian placenta or a part thereof, once collected and prepared generally as above, can be treated in any art-known manner, e.g., can be perfused or disrupted, e.g., digested with one or more tissue-disrupting enzymes, to obtain stem cells.

### Example 2: Physical Disruption and Enzymatic Digestion of Placental Tissue

In one embodiment, stem cells are collected from a mammalian placenta by physical disruption of part of all of the organ. For example, the placenta, or a portion thereof, may be, e.g., crushed, sheared, minced, diced, chopped, macerated or the like. The tissue can then be cultured to obtain a population of stem cells. Typically, the placental tissue is disrupted using, e.g., in a stem cell collection composition, as provided elsewhere herein.

The placenta can be dissected into components prior to physical disruption and/or enzymatic digestion and stem cell recovery. Postpartum cells can be obtained from all or a portion of the amniotic membrane, chorion, umbilical cord, placental cotyledons, or any combination thereof, including from a whole placenta. Postpartum cells can be obtained from placental tissue comprising amnion and chorion. Typically, postpartum cells can be obtained by disruption of a small block of placental tissue, e.g., a block of placental tissue that is about 1, 2, 3, 4, 5, 6, 7, 8, 9, 10, 20, 30, 40, 50, 60, 70, 80, 90, 100, 200, 300, 400, 500, 600, 700, 800, 900 or about 1000 cubic millimeters in volume. Any method of physical disruption can be used, provided that the method of disruption leaves a plurality, or even a majority, such as at least 60%, 70%, 80%, 90%, 95%, 98%, or 99% of the cells in said organ viable, as determined by, e.g., trypan blue exclusion.

Stem cells can generally be collected from a placenta, or portion thereof, at any time within about the first three days post-expulsion, such as between about 8 hours and about 18 hours post-expulsion.

In another specific embodiment, stem cells are collected by physical disruption of placental tissue, wherein the physical disruption includes enzymatic digestion, which can be accomplished by use of one or more tissue-digesting enzymes. The placenta, or a portion thereof, may also be physically disrupted and digested with one or more enzymes, and the resulting material then immersed in, or mixed into, a stem cell collection composition.

An exemplary stem cell collection composition comprises one or more tissue-disruptive enzyme(s). Enzymatic digestion can use a combination of enzymes, e.g., a combination of a matrix metalloprotease and a neutral protease, for example, a combination of collagenase and dispase. In one embodiment, enzymatic digestion of placental tissue uses a combination of a matrix metalloprotease, a neutral protease, and a mucolytic enzyme for digestion of hyaluronic acid, such as a combination of collagenase, dispase, and hyaluronidase or a combination of LIBERASE™ (Boehringer Mannheim Corp., Indianapolis, Ind.) and hyaluronidase. Other enzymes that can be used to disrupt placenta tissue include papain, deoxyribonucleases, serine proteases, such as trypsin, chymotrypsin, or elastase. Serine proteases may be inhibited by alpha 2 microglobulin in serum and therefore the medium used for digestion is usually serum-free. EDTA and DNase are commonly used in enzyme digestion procedures to increase the efficiency of cell recovery. The digestate can be diluted so as to avoid trapping stem cells within the viscous digest.

Any combination of tissue digestion enzymes can be used. Typical concentrations for tissue digestion enzymes include, e.g., 50-200 U/mL for collagenase I and collagenase IV, 1-10 U/mL for dispase, and 10-100 U/mL for elastase. Proteases can be used in combination, that is, two or more proteases in the same digestion reaction, or can be used sequentially in order to liberate postpartum cells. For example, in one embodiment, a placenta, or part thereof, is digested first with an appropriate amount of collagenase I at about 1 to about 2 mg/ml for, e.g., 30 minutes, followed by digestion with trypsin, at a concentration of about 0.25%, for, e.g., 10 minutes, at 37° C. Serine proteases can be used consecutively following use of other enzymes.

In another embodiment, the tissue can further be disrupted by the addition of a chelator, e.g., ethylene glycol bis(2-aminoethyl ether)-N,N,N′N′-tetraacetic acid (EGTA) or ethylenediaminetetraacetic acid (EDTA) to the stem cell collection composition comprising the stem cells, or to a solution in which the tissue is disrupted and/or digested prior to isolation of the stem cells with the stem cell collection composition.

In one embodiment, a digestion can proceed as follows. Approximately a gram of placental tissue is obtained and minced. The tissue is digested in 10 mL of a solution comprising about 1 mg/mL collagenase 1A and about 0.25% trypsin at 37° C. in a shaker at about 100 RPM. The digestate is washed three times with culture medium, and the washed cells are seeded into 2 T-75 flasks. The cells are then isolated by differential adherence, and characterized for, e.g., viability, cell surface markers, differentiation, and the like.

It will be appreciated that where an entire placenta, or portion of a placenta comprising both fetal and maternal cells (for example, where the portion of the placenta comprises the chorion or cotyledons), the postpartum cells collected will comprise a mix of postpartum cells derived from both fetal and maternal sources. Where a portion of the placenta that comprises no, or a negligible number of, maternal cells (for example, amnion), the postpartum cells collected will comprise almost exclusively fetal postpartum cells.

Stem cells can be isolated from disrupted tissue by differential trypsinization followed by culture in one or more new culture containers in fresh proliferation medium, optionally followed by a second differential trypsinization step.

### Example 3: Placental Perfusion

Postpartum cells can also be obtained by perfusion of the mammalian placenta. Methods of perfusing mammalian placenta to obtain stem cells are disclosed, e.g., in Hariri, U.S. Application Publication No. 2002/0123141, and in related U.S. Provisional Application No. 60/754,969, entitled “Improved Medium for Collecting PDSC and Preserving Organs,” filed on Dec. 29, 2005.

Postpartum cells can be collected by perfusion, e.g., through the placental vasculature, using, e.g., a stem cell collection composition as a perfusion solution. In one embodiment, a mammalian placenta is perfused by passage of perfusion solution through either or both of the umbilical artery and umbilical vein. The flow of perfusion solution through the placenta may be accomplished using, e.g., gravity flow into the placenta. The perfusion solution can be forced through the placenta using a pump, e.g., a peristaltic pump. The umbilical vein can be, e.g., cannulated with a cannula, e.g., a TEFLON™ or plastic cannula, that is connected to a sterile connection apparatus, such as sterile tubing. The sterile connection apparatus is connected to a perfusion manifold.

In preparation for perfusion, the placenta can be oriented (e.g., suspended) in such a manner that the umbilical artery and umbilical vein are located at the highest point of the placenta. The placenta can be perfused by passage of a perfusion fluid through the placental vasculature and surrounding tissue. The placenta can also be perfused by passage of a perfusion fluid into the umbilical vein and collection from the umbilical arteries, or passage of a perfusion fluid into the umbilical arteries and collection from the umbilical vein.

In one embodiment, for example, the umbilical artery and the umbilical vein are connected simultaneously, e.g., to a pipette that is connected via a flexible connector to a reservoir of the perfusion solution. The perfusion solution is passed into the umbilical vein and artery. The perfusion solution exudes from and/or passes through the walls of the blood vessels into the surrounding tissues of the placenta, and is collected in a suitable open vessel from the surface of the placenta that was attached to the uterus of the mother during gestation. The perfusion solution may also be introduced through the umbilical cord opening and allowed to flow or percolate out of openings in the wall of the placenta which interfaced with the maternal uterine wall. Placental cells that are collected by this method, which can be referred to as a “pan” method, are typically a mixture of fetal and maternal cells.

In another embodiment, the perfusion solution is passed through the umbilical veins and collected from the umbilical artery, or is passed through the umbilical artery and collected from the umbilical veins. Placental cells collected by this method, which can be referred to as a “closed circuit” method, are typically almost exclusively fetal.

It will be appreciated that perfusion using the pan method, that is, whereby perfusate is collected after it has exuded from the maternal side of the placenta, results in a mix of fetal and maternal cells. As a result, the cells collected by this method comprise a mixed population of postpartum cells of both fetal and maternal origin. In contrast, perfusion solely through the placental vasculature in the closed circuit method, whereby perfusion fluid is passed through one or two placental vessels and is collected solely through the remaining vessel(s), results in the collection of a population of postpartum cells almost exclusively of fetal origin.

The closed circuit perfusion method can, in one embodiment, be performed as follows. A post-partum placenta is obtained within about 48 hours after birth. The umbilical cord is clamped and cut above the clamp. The umbilical cord can be discarded, or can processed to recover, e.g., umbilical cord stem cells, and/or to process the umbilical cord membrane for the production of a biomaterial. The amniotic membrane can be retained during perfusion, or can be separated from the chorion, e.g., using blunt dissection with the fingers. If the amniotic membrane is separated from the chorion prior to perfusion, it can be, e.g., discarded, or processed, e.g., to obtain stem cells by enzymatic digestion, or to produce, e.g., an amniotic membrane biomaterial, e.g., the biomaterial described in U.S. Application Publication No. 2004/0048796. After cleaning the placenta of all visible blood clots and residual blood, e.g., using sterile gauze, the umbilical cord vessels are exposed, e.g., by partially cutting the umbilical cord membrane to expose a cross-section of the cord. The vessels are identified, and opened, e.g., by advancing a closed alligator clamp through the cut end of each vessel. The apparatus, e.g., plastic tubing connected to a perfusion device or peristaltic pump, is then inserted into each of the placental arteries. The pump can be any pump suitable for the purpose, e.g., a peristaltic pump. Plastic tubing, connected to a sterile collection reservoir, e.g., a blood bag such as a 250 mL collection bag, is then inserted into the placental vein. Alternatively, the tubing connected to the pump is inserted into the placental vein, and tubes to a collection reservoir(s) are inserted into one or both of the placental arteries. The placenta is then perfused with a volume of perfusion solution, e.g., about 750 mL of perfusion solution. Cells in the perfusate are then collected, e.g., by centrifugation.

In one embodiment, the proximal umbilical cord is clamped during perfusion, such as, within 4-5 cm of the cord's insertion into the placental disc.

The first collection of perfusion fluid from a mammalian placenta during the exsanguination process is generally colored with residual red blood cells of the cord blood and/or placental blood. The perfusion fluid becomes more colorless as perfusion proceeds and the residual cord blood cells are washed out of the placenta. Generally from 30 to 100 mL (milliliter) of perfusion fluid is adequate to initially exsanguinate the placenta, but more or less perfusion fluid may be used depending on the observed results.

The volume of perfusion liquid used to collect postpartum cells may vary depending upon the number of stem cells to be collected, the size of the placenta, the number of collections to be made from a single placenta, etc. In various embodiments, the volume of perfusion liquid may be from 50 mL to 5000 mL, 50 mL to 4000 mL, 50 mL to 3000 mL, 100 mL to 2000 mL, 250 mL to 2000 mL, 500 mL to 2000 mL, or 750 mL to 2000 mL. Typically, the placenta is perfused with 700-800 mL of perfusion liquid following exsanguination.

The placenta can be perfused a plurality of times over the course of several hours or several days. Where the placenta is to be perfused a plurality of times, it may be maintained or cultured under aseptic conditions in a container or other suitable vessel, and perfused with the stem cell collection composition, or a standard perfusion solution (e.g., a normal saline solution such as phosphate buffered saline (“PBS”)) with or without an anticoagulant (e.g., heparin, warfarin sodium, coumarin, bishydroxycoumarin), and/or with or without an antimicrobial agent (e.g., β-mercaptoethanol (0.1 mM); antibiotics such as streptomycin (e.g., at 40-100 μg/mL), penicillin (e.g., at 40 U/mL), amphotericin B (e.g., at 0.5 μg/mL). In one embodiment, an isolated placenta is maintained or cultured for a period of time without collecting the perfusate, such that the placenta is maintained or cultured for 1, 2, 3, 4, 5, 6, 7, 8, 9, 10, 11, 12, 13, 14, 15, 16, 17, 18, 19, 20, 21, 22, 23, or 24 hours, or 2 or 3 or more days before perfusion and collection of perfusate. The perfused placenta can be maintained for one or more additional time(s), e.g., 1, 2, 3, 4, 5, 6, 7, 8, 9, 10, 11, 12, 13, 14, 15, 16, 17, 18, 19, 20, 21, 22, 23, 24 or more hours, and perfused a second time with, e.g., 700-800 mL perfusion fluid. The placenta can be perfused 1, 2, 3, 4, 5 or more times, for example, once every 1, 2, 3, 4, 5 or 6 hours. In one embodiment, perfusion of the placenta and collection of perfusion solution, e.g., stem cell collection composition, is repeated until the number of recovered nucleated cells falls below 100 cells/ml. The perfusates at different time points can be further processed individually to recover time-dependent populations of cells, e.g., stem cells. Perfusates from different time points can also be pooled. In a specific embodiment, stem cells are collected at a time or times between about 8 hours and about 18 hours post-expulsion.

Without wishing to be bound by any theory, after exsanguination and a sufficient time of perfusion of the placenta, postpartum cells are believed to migrate into the exsanguinated and perfused microcirculation of the placenta where, according to methods provided herein, they are collected, such as by washing into a collecting vessel by perfusion. Perfusing the isolated placenta not only serves to remove residual cord blood but also provide the placenta with the appropriate nutrients, including oxygen. The placenta may be cultivated and perfused with a similar solution which was used to remove the residual cord blood cells, e.g., without the addition of anticoagulant agents.

Perfusion according to the methods provided herein can result in the collection of significantly more postpartum cells than the number obtainable from a mammalian placenta not perfused with said solution, and not otherwise treated to obtain stem cells (e.g., by tissue disruption, e.g., enzymatic digestion). In this context, “significantly more” means at least 10% more. Perfusion according to the methods provided herein can yield significantly more postpartum cells than, e.g., the number of postpartum cells obtainable from culture medium in which a placenta, or portion thereof, has been cultured.

Stem cells can be isolated from placenta by perfusion with a solution comprising one or more proteases or other tissue-disruptive enzymes. In a specific embodiment, a placenta or portion thereof (e.g., amniotic membrane, amnion and chorion, placental lobule or cotyledon, umbilical cord, or combination of any of the foregoing) is brought to 25-37° C., and is incubated with one or more tissue-disruptive enzymes in 200 mL of a culture medium for 30 minutes. Cells from the perfusate are collected, brought to 4° C., and washed with a cold inhibitor mix comprising 5 mM EDTA, 2 mM dithiothreitol and 2 mM beta-mercaptoethanol. The stem cells are washed after several minutes with a cold (e.g., 4° C.) stem cell collection composition.

### Example 4: Isolation, Sorting, and Characterization of Postpartum Cells

Cells from mammalian postpartum tissue, whether obtained by perfusion or enzymatic digestion, can initially be purified from (i.e., be isolated from) other cells by Ficoll® gradient centrifugation. Such centrifugation can follow any standard protocol for centrifugation speed, etc. In one embodiment, for example, cells collected from the placenta are recovered from perfusate by centrifugation at 5000×g for 15 minutes at room temperature, which separates cells from, e.g., contaminating debris and platelets. In another embodiment, placental perfusate is concentrated to about 200 ml, gently layered over Ficoll, and centrifuged at about 1100×g for 20 minutes at 22° C., and the low-density interface layer of cells is collected for further processing.

Cell pellets can be resuspended in fresh stem cell collection composition, or a medium suitable for stem cell maintenance, e.g., IMDM serum-free medium containing 2 U/mL heparin and 2 mM EDTA (GibcoBRL, NY). The total mononuclear cell fraction can be isolated, e.g., using Lymphoprep™ (Nycomed Pharma, Oslo, Norway) according to the manufacturer's recommended procedure.

As used herein, “isolating” postpartum cells means to remove at least 20%, 30%, 40%, 50%, 60%, 70%, 80%, 90%, 95% or 99% of the cells with which the stem cells are normally associated in the intact mammalian placenta. A stem cell from an organ is “isolated” when it is present in a population of cells that comprises fewer than 50% of the cells with which the stem cell is normally associated in the intact organ.

Placental cells obtained by perfusion or digestion can, for example, be further, or initially, isolated by differential trypsinization using, e.g., a solution of 0.05% trypsin with 0.2% EDTA (Sigma, St. Louis, Mo.). Differential trypsinization is possible because certain placenta derived stem cells (PDSC) typically detach from plastic surfaces within about five minutes whereas other adherent populations typically require more than 20-30 minutes incubation. The detached PDSC can be harvested following trypsinization and trypsin neutralization, using, e.g., Trypsin Neutralizing Solution (TNS, Cambrex). In one embodiment of isolation of adherent cells, aliquots of, for example, about 5-10×10⁶ cells are placed in each of several T-75 flasks, such as fibronectin-coated T75 flasks. In such an embodiment, the cells can be cultured with commercially available Mesenchymal Stem Cell Growth Medium (MSCGM) (Cambrex), and placed in a tissue culture incubator (37° C., 5% CO₂). After 10 to 15 days, non-adherent cells are removed from the flasks by washing with PBS. The PBS is then replaced by MSCGM. Flasks can be examined daily for the presence of various adherent cell types and in particular, for identification and expansion of clusters of fibroblastoid cells.

The number and type of cells collected from a mammalian placenta can be monitored, for example, by measuring changes in morphology and cell surface markers using standard cell detection techniques such as flow cytometry, cell sorting, immunocytochemistry (e.g., staining with tissue specific or cell-marker specific antibodies) fluorescence activated cell sorting (FACS), magnetic activated cell sorting (MACS), by examination of the morphology of cells using light or confocal microscopy, and/or by measuring changes in gene expression using techniques well known in the art, such as PCR and gene expression profiling. In specific embodiments, the technique is flow cytometry. In other specific embodiments, the technique is FACS. These techniques can be used, too, to identify cells that are positive for one or more particular markers. For example, using antibodies to CD34, one can determine, using the techniques above, whether a cell comprises a detectable amount of CD34; if so, the cell is CD34⁺. Likewise, if a cell produces enough OCT-4 RNA to be detectable by RT-PCR, or significantly more OCT-4 RNA than an adult cell, the cell is OCT-4⁺ Antibodies to cell surface markers (e.g., CD markers such as CD34) and the sequence of stem cell-specific genes, such as OCT-4, are well-known in the art.

Placental cells, particularly cells that have been isolated by Ficoll® separation, differential adherence, or a combination of both, may be sorted using a fluorescence activated cell sorter (FACS). Fluorescence activated cell sorting (FACS) is a well-known method for separating particles, including cells, based on the fluorescent properties of the particles (Kamarch, Methods Enzymol 1987, 151:150-165). Laser excitation of fluorescent moieties in the individual particles results in a small electrical charge allowing electromagnetic separation of positive and negative particles from a mixture. In one embodiment, cell surface marker-specific antibodies or ligands are labeled with distinct fluorescent labels. Cells are processed through the cell sorter, allowing separation of cells based on their ability to bind to the antibodies used. FACS sorted particles may be directly deposited into individual wells of 96-well or 384-well plates to facilitate separation and cloning.

In one sorting scheme, stem cells from placenta are sorted on the basis of expression of the markers CD34, CD38, CD44, CD45, CD73, CD105, OCT-4 and/or HLA-G. This can be accomplished in connection with procedures to select stem cells on the basis of their adherence properties in culture. For example, an adherence selection stem can be accomplished before or after sorting on the basis of marker expression. In one embodiment, for example, cells are sorted first on the basis of their expression of CD34; CD34− cells are retained, and cells that are CD200+HLA-G+, are separated from all other CD34− cells. In another embodiment, cells from placenta are based on their expression of markers CD200 and/or HLA-G; for example, cells displaying either of these markers are isolated for further use. Cells that express, e.g., CD200 and/or HLA-G can, in a specific embodiment, be further sorted based on their expression of CD73 and/or CD105, or epitopes recognized by antibodies SH2, SH3 or SH4, or lack of expression of CD34, CD38 or CD45. For example, in one embodiment, placental cells are sorted by expression, or lack thereof, of CD200, HLA-G, CD73, CD105, CD34, CD38 and CD45, and placental cells that are CD200+, HLA-G+, CD73+, CD105+, CD34−, CD38− and CD45− are isolated from other placental cells for further use.

With respect to antibody-mediated detection and sorting of postpartum cells, any antibody, specific for a particular marker, can be used, in combination with any fluorophore or other label suitable for the detection and sorting of cells (e.g., fluorescence-activated cell sorting). Antibody/fluorophore combinations to specific markers include, but are not limited to, fluorescein isothiocyanate (FITC) conjugated monoclonal antibodies against HLA-G (available from Serotec, Raleigh, N.C.), CD10 (available from BD Immunocytometry Systems, San Jose, Calif.), CD44 (available from BD Biosciences Pharmingen, San Jose, Calif.), and CD105 (available from R&D Systems Inc., Minneapolis, Minn.); phycoerythrin (PE) conjugated monoclonal antibodies against CD44, CD200, CD117, and CD13 (BD Biosciences Pharmingen); phycoerythrin-Cy7 (PE Cy7) conjugated monoclonal antibodies against CD33 and CD10 (BD Biosciences Pharmingen); allophycocyanin (APC) conjugated streptavidin and monoclonal antibodies against CD38 (BD Biosciences Pharmingen); and Biotinylated CD90 (BD Biosciences Pharmingen). Other antibodies that can be used include, but are not limited to, CD133-APC (Miltenyi), KDR-Biotin (CD309, Abcam), CytokeratinK-Fitc (Sigma or Dako), HLA ABC-Fitc (BD), HLA DRDQDP-PE (BD), beta-2-microglobulin-PE (BD), CD80-PE (BD) and CD86-APC (BD).

Other antibody/label combinations that can be used include, but are not limited to, CD45-PerCP (peridin chlorophyll protein); CD44-PE; CD19-PE; CD10-F (fluorescein); HLA-G-F and 7-amino-actinomycin-D (7-AAD); HLA-ABC-F; and the like.

Postpartum cells can be assayed for CD117 or CD133 using, for example, phycoerythrin-Cy5 (PE Cy5) conjugated streptavidin and biotin conjugated monoclonal antibodies against CD117 or CD133; however, using this system, the cells can appear to be positive for CD117 or CD133, respectively, because of a relatively high background.

Postpartum cells can be labeled with an antibody to a single marker and detected and/sorted. Postpartum cells can also be simultaneously labeled with multiple antibodies to different markers.

In another embodiment, magnetic beads can be used to separate cells. The cells may be sorted using a magnetic activated cell sorting (MACS) technique, a method for separating particles based on their ability to bind magnetic beads (0.5-100 μm diameter). A variety of useful modifications can be performed on the magnetic microspheres, including covalent addition of antibody that specifically recognizes a particular cell surface molecule or hapten. The beads are then mixed with the cells to allow binding. Cells are then passed through a magnetic field to separate out cells having the specific cell surface marker. In one embodiment, these cells can then be isolated and re-mixed with magnetic beads coupled to an antibody against additional cell surface markers. The cells are again passed through a magnetic field, isolating cells that bound both the antibodies. Such cells can then be diluted into separate dishes, such as microtiter dishes for clonal isolation.

PDSC can also be characterized and/or sorted based on cell morphology and growth characteristics. For example, PDSC can be characterized as having, and/or selected on the basis of, e.g., a fibroblastoid appearance in culture. PDSC can also be characterized as having, and/or be selected, on the basis of their ability to form embryoid-like bodies. In one embodiment, for example, placental cells that are fibroblastoid in shape, express CD73 and CD105, and produce one or more embryoid-like bodies in culture are isolated from other placental cells. In another embodiment, OCT-4+ placental cells that produce one or more embryoid-like bodies in culture are isolated from other placental cells.

In another embodiment, postpartum cells can be identified and characterized by a colony forming unit assay. Colony forming unit assays are commonly known in the art, such as MESEN CULT™ medium (Stem Cell Technologies, Inc., Vancouver, British Columbia).

Postpartum cells can be assessed for viability, proliferation potential, and longevity using standard techniques known in the art, such as trypan blue exclusion assay, fluorescein diacetate uptake assay, propidium iodide uptake assay (to assess viability); and thymidine uptake assay, MTT cell proliferation assay (to assess proliferation). Longevity may be determined by methods well known in the art, such as by determining the maximum number of population doubling in an extended culture.

Selected postpartum cells can also be separated from other placental cells using other techniques known in the art, e.g., selective growth of desired cells (positive selection), selective destruction of unwanted cells (negative selection); separation based upon differential cell agglutinability in the mixed population as, for example, with soybean agglutinin; freeze-thaw procedures; filtration; conventional and zonal centrifugation; centrifugal elutriation (counter-streaming centrifugation); unit gravity separation; countercurrent distribution; electrophoresis; and the like.

### Example 5: Generation and Characterization of iPS Cells

Postpartum cells are collected and characterized as described above. Generation of iPS cells is compared between cord blood mononuclear cells, hematopoietic progenitor cells, and placenta derived stem cells.

Selected factors are introduced to the cells as described in the embodiments, in one example by lentiviral transduction and cells are allowed to form colonies. Colonies with embryonic stem (ES) cell-like morphology are selected and analyzed for ES cell surface markers as described above for postpartum cells. Cells positive for desired surface markers are further analyzed for expression of ES cell markers by RT-PCR.

Cell populations expressing both the desired ES cell surface markers and the desired ES cell expression markers are further analyzed to verify pluripotency in an in vivo model. Briefly, the putative human iPS cells are injected into mice and tumors allowed to form. After tumor formation, said tumors are analyzed for the presence of each of mesoderm, endoderm and ectoderm and putative human iPS cell populations which form tumors comprising cells of all three germ layers are identified as iPS cells.

### Example 6: Preservation of PDSC

IPS cells can be preserved, that is, placed under conditions that allow for long-term storage, or conditions that inhibit cell death by, e.g., apoptosis or necrosis.

IPS cells can be preserved using, e.g., a composition comprising an apoptosis inhibitor, necrosis inhibitor and/or an oxygen-carrying perfluorocarbon, as described in related U.S. Provisional Application No. 60/754,969, entitled “Improved Medium for Collecting iPS cells and Preserving Organs,” filed on Dec. 25, 2005. In one embodiment, provided is a method of preserving a population of stem cells comprising contacting said population of stem cells with a stem cell collection composition comprising an inhibitor of apoptosis and an oxygen-carrying perfluorocarbon, wherein said inhibitor of apoptosis is present in an amount and for a time sufficient to reduce or prevent apoptosis in the population of stem cells, as compared to a population of stem cells not contacted with the inhibitor of apoptosis. In a specific embodiment, said inhibitor of apoptosis is a caspase inhibitor. In another specific embodiment, said inhibitor of apoptosis is a JNK inhibitor. In a more specific embodiment, said JNK inhibitor does not modulate differentiation or proliferation of said stem cells. In another embodiment, said stem cell collection composition comprises said inhibitor of apoptosis and said oxygen-carrying perfluorocarbon in separate phases. In another embodiment, said stem cell collection composition comprises said inhibitor of apoptosis and said oxygen-carrying perfluorocarbon in an emulsion. In another embodiment, the stem cell collection composition additionally comprises an emulsifier, e.g., lecithin. In another embodiment, said apoptosis inhibitor and said perfluorocarbon are between about 0 C and about 25 C at the time of contacting the stem cells. In another more specific embodiment, said apoptosis inhibitor and said perfluorocarbon are between about 2 C and 10 C, or between about 2 C and about 5 C, at the time of contacting the stem cells. In another more specific embodiment, said contacting is performed during transport of said population of stem cells. In another more specific embodiment, said contacting is performed during freezing and thawing of said population of stem cells.

In another embodiment, provided is a method of preserving a population of iPS cells comprising contacting said population of stem cells with an inhibitor of apoptosis and an organ-preserving compound, wherein said inhibitor of apoptosis is present in an amount and for a time sufficient to reduce or prevent apoptosis in the population of stem cells, as compared to a population of stem cells not contacted with the inhibitor of apoptosis. In a specific embodiment, the organ-preserving compound is UW solution (described in U.S. Pat. No. 4,798,824; also known as ViaSpan®; see also Southard et al., Transplantation 1990, 49(2):251-257) or a solution described in Stern et al., U.S. Pat. No. 5,552,267. In another embodiment, said organ-preserving compound is hydroxyethyl starch, lactobionic acid, raffinose, or a combination thereof. In another embodiment, the stem cell collection composition additionally comprises an oxygen-carrying perfluorocarbon, either in two phases or as an emulsion.

In another embodiment of the method, iPS cells are contacted with a stem cell collection composition comprising an apoptosis inhibitor and oxygen-carrying perfluorocarbon, organ-preserving compound, or combination thereof, during perfusion. In another embodiment, said stem cells are contacted during a process of tissue disruption, e.g., enzymatic digestion. In another embodiment, iPS cells are contacted with said stem cell collection compound after collection by perfusion, or after collection by tissue disruption, e.g., enzymatic digestion.

Typically, during placental cell collection, enrichment and isolation, cell stress due to hypoxia and mechanical stress is minimized or eliminated. In another embodiment of the method, therefore, a stem cell, or population of stem cells, is exposed to a hypoxic condition during collection, enrichment or isolation for less than six hours during said preservation, wherein a hypoxic condition is a concentration of oxygen that is less than normal blood oxygen concentration. In a more specific embodiment, said population of stem cells is exposed to said hypoxic condition for less than two hours during said preservation. In another more specific embodiment, said population of stem cells is exposed to said hypoxic condition for less than one hour, or less than thirty minutes, or is not exposed to a hypoxic condition, during collection, enrichment or isolation. In another specific embodiment, said population of stem cells is not exposed to shear stress during collection, enrichment or isolation.

The iPS cells provided herein can be cryopreserved, e.g., in cryopreservation medium in small containers, e.g., ampoules. Suitable cryopreservation medium includes, but is not limited to, culture medium including, e.g., growth medium, or cell freezing medium, for example commercially available cell freezing medium, e.g., C2695, C2639 or C6039 (Sigma). Cryopreservation medium can comprise DMSO (dimethylsulfoxide), at a concentration of, e.g., about 10% (v/v). Cryopreservation medium may comprise additional agents, for example, methylcellulose and/or glycerol. PDSC can be cooled at about 1 C/min during cryopreservation. An exemplary cryopreservation temperature is about −80 C to about −180 C, such as about −125 C to about −140° C. Cryopreserved cells can be transferred to liquid nitrogen prior to thawing for use. In some embodiments, for example, once the ampoules have reached about −90 C, they are transferred to a liquid nitrogen storage area. Cryopreservation can also be done using a controlled-rate freezer. Cryopreserved cells can be thawed at a temperature of about 25 C to about 40 C, such as to a temperature of about 37 C.

### Preparation of Pharmaceutical Compositions

Populations of isolated iPS cells or populations of cells comprising the isolated iPS cells, can be formulated into pharmaceutical compositions for use in vivo, e.g., in the methods provided herein. Such pharmaceutical compositions comprise a population of isolated placental cells, or a population of cells comprising isolated placental cells, in a pharmaceutically-acceptable carrier, e.g., a saline solution or other accepted physiologically-acceptable solution for in vivo administration. Pharmaceutical compositions comprising the isolated placental cells described herein can comprise any, or any combination, of the isolated placental cell populations, or isolated placental cells, described elsewhere herein. The pharmaceutical compositions can comprise fetal, maternal, or both fetal and maternal isolated placental cells. The pharmaceutical compositions provided herein can further comprise isolated placental cells obtained from a single individual or placenta, or from a plurality of individuals or placentae.

The pharmaceutical compositions provided herein can comprise any number of isolated placental cells. For example, a single unit dose of isolated placental cells can comprise, in various embodiments, about, at least, or no more than 1×105, 5×105, 1×106, 5×106, 1×107, 5×107, 1×108, 5×108, 1×109, 5×109, 1×1010, 5×1010, 1×1011 or more isolated placental cells.

The pharmaceutical compositions provided herein comprise populations of cells that comprise 50% viable cells or more (that is, at least 50% of the cells in the population are functional or living). In certain embodiments, at least 60% of the cells in the population are viable. In a specific embodiment, at least 70%, 80%, 90%, 95%, or 99% of the cells in the population in the pharmaceutical composition are viable.

The pharmaceutical compositions provided herein can comprise one or more compounds that, e.g., facilitate engraftment (e.g., anti-T-cell receptor antibodies, an immunosuppressant, or the like); stabilizers such as albumin, dextran 40, gelatin, hydroxyethyl starch, plasmalyte, and the like.

When formulated as an injectable solution, in one embodiment, the pharmaceutical composition comprises about 1% to 1.5% HSA and about 2.5% dextran. In one embodiment, the pharmaceutical composition comprises from about 5×106 cells per milliliter to about 2×107 cells per milliliter in a solution comprising 5% HSA and 10% dextran, optionally comprising an immunosuppressant, e.g., cyclosporine A at, e.g., 10 mg/kg.

In other embodiments, the pharmaceutical composition, e.g., a solution, comprises a plurality of cells, e.g., isolated placental cells, for example, PDSC, wherein said pharmaceutical composition comprises between about 1.0±0.3×106 cells per milliliter to about 5.0±1.5×106 cells per milliliter. In other embodiments, the pharmaceutical composition comprises between about 1.5×106 cells per milliliter to about 3.75×106 cells per milliliter. In other embodiments, the pharmaceutical composition comprises between about 1×106 cells/mL to about 50×106 cells/mL, about 1×106 cells/mL to about 40×106 cells/mL, about 1×106 cells/mL to about 30×106 cells/mL, about 1×106 cells/mL to about 20×106 cells/mL, about 1×106 cells/mL to about 15×106 cells/mL, or about 1×106 cells/mL to about 10×106 cells/mL. In certain embodiments, the pharmaceutical composition comprises no visible cell clumps (i.e., no macro cell clumps), or substantially no such visible clumps. As used herein, “macro cell clumps” means an aggregation of cells visible without magnification, e.g., visible to the naked eye, and generally refers to a cell aggregation larger than about 150 micron. In some embodiments, the pharmaceutical composition comprises about 2.5%, 3.0%, 3.5%, 4.0%, 4.5%, 5.0%, 5.5%, 6.0%, 6.5%, 7.0%, 7.5% 8.0%, 8.5%, 9.0%, 9.5% or 10% dextran, e.g., dextran-40. In a specific embodiment, said composition comprises about 7.5% to about 9% dextran-40. In a specific embodiment, said composition comprises about 5.5% dextran-40. In certain embodiments, the pharmaceutical composition comprises from about 1% to about 15% human serum albumin (HSA). In specific embodiments, the pharmaceutical composition comprises about 1%, 2%, 3%, 4%, 5%, 6%, 7%, 8%, 9%, 10%, 11%, 12%, 13%, 14% or 15% HSA. In a specific embodiment, said cells have been cryopreserved and thawed. In another specific embodiment, said cells have been filtered through a 70 μM to 100 μM filter. In another specific embodiment, said composition comprises no visible cell clumps. In another specific embodiment, said composition comprises fewer than about 200 cell clumps per 106 cells, wherein said cell clumps are visible only under a microscope, e.g., a light microscope. In another specific embodiment, said composition comprises fewer than about 150 cell clumps per 106 cells, wherein said cell clumps are visible only under a microscope, e.g., a light microscope. In another specific embodiment, said composition comprises fewer than about 100 cell clumps per 106 cells, wherein said cell clumps are visible only under a microscope, e.g., a light microscope.

In a specific embodiment, the pharmaceutical composition comprises about 1.0±0.3 ×106 cells per milliliter, about 5.5% dextran-40 (w/v), about 10% HSA (w/v), and about 5% DMSO (v/v).

In other embodiments, the pharmaceutical composition comprises a plurality of cells, e.g., a plurality of isolated placental cells in a solution comprising 10% dextran-40, wherein the pharmaceutical composition comprises between about 1.0±0.3×10⁶ cells per milliliter to about 5.0±1.5×10⁶ cells per milliliter, and wherein said composition comprises no cell clumps visible with the unaided eye (i.e., comprises no macro cell clumps). In some embodiments, the pharmaceutical composition comprises between about 1.5×10⁶ cells per milliliter to about 3.75×106 cells per milliliter. In a specific embodiment, said cells have been cryopreserved and thawed. In another specific embodiment, said cells have been filtered through a 70 μM to 100 μM filter. In another specific embodiment, said composition comprises fewer than about 200 micro cell clumps (that is, cell clumps visible only with magnification) per 10⁶ cells. In another specific embodiment, the pharmaceutical composition comprises fewer than about 150 micro cell clumps per 106 cells. In another specific embodiment, the pharmaceutical composition comprises fewer than about 100 micro cell clumps per 10⁶ cells. In another specific embodiment, the pharmaceutical composition comprises less than 15%, 14%, 13%, 12%, 11%, 10%, 9%, 8%, 7%, 6%, 5%, 4%, 3%, or 2% DMSO, or less than 1%, 0.9%, 0.8%, 0.7%, 0.6%, 0.5%, 0.4%, 0.3%, 0.2%, or 0.1% DMSO.

Further provided herein are compositions comprising cells, wherein said compositions are produced by one of the methods disclosed herein. For example, in one embodiment, the pharmaceutical composition comprises cells, wherein the pharmaceutical composition is produced by a method comprising filtering a solution comprising PDSC, to form a filtered cell-containing solution; diluting the filtered cell-containing solution with a first solution to about 1 to 50×10⁶, 1 to 40×10⁶, 1 to 30×10⁶, 1 to 20×10⁶, 1 to 15×10⁶, or 1 to 10×10⁶ cells per milliliter, e.g., prior to cryopreservation; and diluting the resulting filtered cell-containing solution with a second solution comprising dextran, but not comprising human serum albumin (HSA) to produce said composition. In certain embodiments, said diluting is to no more than about 15×10⁶ cells per milliliter. In certain embodiments, said diluting is to no more than about 10±3×10⁶ cells per milliliter. In certain embodiments, said diluting is to no more than about 7.5×10⁶ cells per milliliter. In other certain embodiments, if the filtered cell-containing solution, prior to the dilution, comprises less than about 15×10⁶ cells per milliliter, filtration is optional. In other certain embodiments, if the filtered cell-containing solution, prior to the dilution, comprises less than about 10±3×10⁶ cells per milliliter, filtration is optional. In other certain embodiments, if the filtered cell-containing solution, prior to the dilution, comprises less than about 7.5×10⁶ cells per milliliter, filtration is optional.

In a specific embodiment, the cells are cryopreserved between said diluting with a first dilution solution and said diluting with said second dilution solution. In another specific embodiment, the first dilution solution comprises dextran and HSA. The dextran in the first dilution solution or second dilution solution can be dextran of any molecular weight, e.g., dextran having a molecular weight of from about 10 kDa to about 150 kDa. In some embodiments, said dextran in said first dilution solution or said second solution is about 2.5%, 3.0%, 3.5%, 4.0%, 4.5%, 5.0%, 5.5%, 6.0%, 6.5%, 7.0%, 7.5% 8.0%, 8.5%, 9.0%, 9.5% or 10% dextran. In another specific embodiment, the dextran in said first dilution solution or said second dilution solution is dextran-40. In another specific embodiment, the dextran in said first dilution solution and said second dilution solution is dextran-40. In another specific embodiment, said dextran-40 in said first dilution solution is 5.0% dextran-40. In another specific embodiment, said dextran-40 in said first dilution solution is 5.5% dextran-40. In another specific embodiment, said dextran-40 in said second dilution solution is 10% dextran-40. In another specific embodiment, said HSA in said solution comprising HSA is 1 to 15% HSA. In another specific embodiment, said HSA in said solution comprising HSA is about 1%, 2%, 3%, 4%, 5%, 6%, 7%, 8%, 9%, 10%, 11%, 12%, 13%, 14% or 15% HSA. In another specific embodiment, said HSA in said solution comprising HSA is 10% HSA. In another specific embodiment, said first dilution solution comprises HSA. In another specific embodiment, said HSA in said first dilution solution is 10% HSA. In another specific embodiment, said first dilution solution comprises a cryoprotectant. In another specific embodiment, said cryoprotectant is DMSO. In another specific embodiment, said dextran-40 in said second dilution solution is about 10% dextran-40. In another specific embodiment, said composition comprising cells comprises about 7.5% to about 9% dextran. In another specific embodiment, the pharmaceutical composition comprises from about 1.0±0.3×10⁶ cells per milliliter to about 5.0±1.5×10⁶ cells per milliliter. In another specific embodiment, the pharmaceutical composition comprises from about 1.5×10⁶ cells per milliliter to about 3.75×10⁶ cells per milliliter.

Isolated placental cells in the compositions, e.g., pharmaceutical compositions, provided herein, can comprise placental cells derived from a single donor, or from multiple donors. The isolated placental cells can be completely HLA-matched to an intended recipient, or partially or completely HLA-mismatched.

Particular embodiments of this invention are described herein, including the best mode known to the inventors for carrying out the invention. Upon reading the foregoing description, variations of the disclosed embodiments may become apparent to individuals working in the art, and it is expected that those skilled artisans may employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All publications, patent applications, accession numbers, and other references cited in this specification are herein incorporated by reference in its entirety as if each individual publication or patent application were specifically and individually indicated to be incorporated by reference. The publications discussed herein are provided solely for their disclosure prior to the filing date of the present application. Nothing herein is to be construed as an admission that the present invention is not entitled to antedate such publication by virtue of prior invention. Further, the dates of publication provided can be different from the actual publication dates which can need to be independently confirmed.

1. An induced pluripotent stem (iPS) cell, or population of iPS cells, wherein the cell or cells giving rise to the iPS cell(s) are obtained from human postpartum tissue or cells, wherein the iPS cell(s) have increased levels of one or more factors selected from Group I: an Oct family member, a Sox family member, a Klf family member, a Myc family member, Nanog, Lin28, and combinations thereof.

2.-25. (canceled)

26. The iPS cell or population of iPS cells of claim 1, wherein the postpartum cells are obtained from the umbilical cord, preferably wherein the postpartum cells are selected from the group consisting of cord blood, Wharton's jelly, and umbilical cord tissue.

27. The iPS cell or population of iPS cells of claim 1, wherein the postpartum cells are placental blood.

28. The iPS cell or population of iPS cells of claim 1, wherein the postpartum cells are placental perfusate.

29. The iPS cell or population of iPS cells of claim 1, wherein the postpartum tissue is amniotic membrane.

30. The iPS cell or population of iPS cells of claim 1, wherein the postpartum tissue is derived from amniotic fluid.

31. The iPS cell or population of iPS cells of claim 1, wherein the postpartum tissue is chorion.

32. The iPS cell or population of iPS cells of claim 1, wherein the postpartum tissue is decidua.

33.-34. (canceled)

35. The iPS cell or population of iPS cells of claim 1, wherein the postpartum tissue is maternal derived tissue.

36. The iPS cell or population of iPS cells of claim 1, wherein the postpartum tissue is non-maternal derived tissue.

37.-39. (canceled)

40. The iPS cell or population of iPS cells of claim 1, wherein the postpartum tissue comprises cells that are CD34⁺.

41. The iPS cell or population of iPS cells of claim 1, wherein the postpartum tissue comprises cells that are CD34⁻, CD10⁺, SH2⁺, and CD90⁺ placental multipotent cells.

42. The iPS cell or population of iPS cells of claim 1, wherein the postpartum tissue comprises cells that are CD34⁻, CD38⁻, CD45⁻, CD10⁺, CD29⁺, CD44⁺, CD54⁺, CD90⁺, SH2⁺, SH3⁺, SH4⁺ and OCT-4⁺.

43. The iPS cell or population of iPS cells of claim 1, wherein the postpartum tissue comprises cells that are CD45⁻, CD10⁺, CD105⁺, and CD200⁺.

44.-46. (canceled)

47. The iPS cell or population of iPS cells of claim 1, wherein the postpartum tissue comprises cells that are CD34⁺, CD38⁻, CD45⁻, and OCT-4⁺, and CD200⁺.

48. The iPS cell or population of iPS cells of claim 1, wherein the postpartum tissue comprises cells that are CD34⁻, CD38⁻, CD45⁻, CD73⁺, OCT-4⁺, and CD200⁺.

49.-50. (canceled)

51. The iPS cell or population of iPS cells of claim 1, wherein the postpartum tissue comprises cells that are CD73⁺, CD105⁺, and CD200⁺.

52.-53. (canceled)

54. The iPS cell or population of iPS cells of claim 1, wherein the postpartum tissue comprises cells that are CD73⁺, CD105⁺, CD200⁺, and HLA-G⁺.

55. The iPS cell or population of iPS cells of claim 1, wherein the postpartum tissue comprises cells that are CD34−, CD38⁻, CD45⁻, and HLA-G⁺.

56. The iPS cell or population of iPS cells of claim 1, wherein the postpartum tissue comprises cells that are CD34⁻; CD38⁻; CD45⁻ and CD38⁻; CD34⁻ and CD45⁻; CD38⁻ and CD45⁻; CD38⁻ and CD45⁻.

57.-168. (canceled)
